(19) United States
(12) Patent Application Publication
(10) Pub. No.: US 2017/0049591 A1
(43) Pub. Date: Feb. 23, 2017

(54) RADIOACTIVE STENT

(71) Applicant: BOSTON SCIENTIFIC SCIMED, INC., Maple Grove, MN (US)

(72) Inventors: CLAUDE O. CLERC, Marlborough, MA (US); JOHN A. HINGSTON, Framingham, MA (US); MARK RIVARD, Hopkinton, MA (US); ARNOLD M. HERSKOVIC, Chicago, IL (US)

(21) Appl. No.: 15/237,270

(22) Filed: Aug. 15, 2016

Related U.S. Application Data
(60) Provisional application No. 62/206,236, filed on Aug. 17, 2015.

(51) Int. Cl. A61F 2/88

(52) U.S. Cl. CPC A61F 2/88; A61F 2210/0095; A61F 2220/005; A61F 2220/0075

(57) ABSTRACT

This disclosure provides design, material, manufacturing method, and use alternatives for medical devices. An example medical device includes a stent having a plurality of longitudinally extending filaments. The stent also has an inner surface and an outer surface and a plurality of tubular members extending along the stent. Each of the plurality of tubular members is coupled with one or more of the plurality of longitudinally extending filaments and each of the plurality of tubular members is configured to accept a radioactive element, a spacer or both.

### CROSS-REFERENCE TO RELATED APPLICATIONS

This application claims priority under 35 U.S.C. §119 to U.S. Provisional Application Ser. No. 62/206,236, filed Aug. 17, 2015, the entirety of which is incorporated herein by reference.

### TECHNICAL FIELD

The present disclosure pertains to medical devices, and methods for manufacturing medical devices. More particularly, the present disclosure pertains to elongated intracorporeal medical devices including a tubular member connected with other structures, and methods for manufacturing and using such devices.

### BACKGROUND

Some cancers and neoplasms are easier to treat with radiation than others. Hard-to-reach neoplasms, such as those in the esophagus, intestines and other lumens, are often treated via Brachytherapy so as to minimize radiation to adjacent, healthy tissue.

Brachytherapy delivers radiation to small tissue volumes while limiting exposure of healthy tissue. In this regard, the delivered radiation conforms more to the target than any other form of radiation, (including proton therapy) as less normal transient tissue is treated. It features placement of radiation sources, such as small radioactive particles or needles, near or within the target tissue, thus having the advantage over External Beam Radiation Therapy (EBRT) of being more focalized and less damaging to surrounding healthy tissue.

Brachytherapy is a common treatment for esophageal, prostate, and other cancers. Brachytherapy has been used to treat prostate cancer which has been practiced for more than half century. In this situation, very low activity material emitting a low energy is placed next to or within a tumor. Traditionally, these low emitting devices have mostly been left in place permanently except in extraordinary circumstances. It would be desirable to permit the removal and/or replacement of the radioactive material in situ when clinically appropriate, and/or it may be desirable to change the geometry, energy or radioactive sources of the radioactive seeds in situ according to clinical needs. For example, it may be advantageous to replace a depleted radiation source with a new radiation source when clinically necessary to continue radiation therapy and/or it may be advantageous to adjust the position and the activity of the radioactive source on its carrier in response to changes in tumor shape and size, carrier position, and other relevant therapeutic factors.

### BRIEF SUMMARY

This disclosure provides design, material, manufacturing method, and use alternatives for medical devices. An example medical device, comprises:
a stent including a plurality of longitudinally extending filaments, the stent having an inner surface and an outer surface;
a plurality of tubular members extending along the stent;
wherein each of the plurality of tubular members is coupled with one or more of the plurality of longitudinally extending filaments; and
wherein each of the plurality of tubular members is configured to accept a radioactive element, a spacer or both.

Alternatively or additionally to any of the embodiments above, wherein one or more of the plurality of tubular members is interwoven with one or more of the plurality of longitudinally extending filaments.

Alternatively or additionally to any of the embodiments above, wherein the plurality of longitudinally extending filaments are braided together, and wherein at least one of the tubular members is interwoven with the braided filaments.

Alternatively or additionally to any of the embodiments above, wherein one or more of the longitudinally extending filaments and one or more of the plurality of tubular members are braided together.

Alternatively or additionally to any of the embodiments above, wherein the longitudinally extending filaments are braided, and wherein one or more of the plurality of the tubular members extends helically in a clockwise, counter-clockwise or both a clockwise and counter-clockwise direction along the stent.

Alternatively or additionally to any of the embodiments above, wherein the plurality of tubular members includes a first group of tubular members having a first distribution of seeds positioned therein, and wherein the plurality of tubular members includes a second group of tubular members having a second distribution of seeds positioned therein, and where the first and second distributions of seeds are different.

Alternatively or additionally to any of the embodiments above, wherein the first distribution of seeds includes a first seed, and wherein the second distribution of seeds includes a second seed, wherein the first seed is closer to a proximal end of the stent than the second seed.

Alternatively or additionally to any of the embodiments above, wherein the first seed is approximately 5 mm away from the proximal end of the stent and wherein the second seed is approximately 20 mm from the proximal end of the stent.

Alternatively or additionally to any of the embodiments above, wherein at least a portion of the plurality of tubular members extends along the inner surface of the stent.

Alternatively or additionally to any of the embodiments above, wherein at least a portion of the plurality of tubular members extends along the outer surface of the stent.

Alternatively or additionally to any of the embodiments above, wherein at least a portion of the plurality of tubular members extends from the inner stent surface to the outer stent surface through an opening in the stent.

Alternatively or additionally to any of the embodiments above, wherein one or more of the tubular members are sutured to one or more of the longitudinally extending stent filaments.

Alternatively or additionally to any of the embodiments above, wherein the stent has a distal portion having an outer diameter, a proximal portion having an outer diameter substantially equal to the distal portion outer diameter, and an intermediate portion located between the distal and proximal portions, wherein the intermediate portion has an outer diameter less than the outer diameter of the proximal and distal portions, and wherein the tubular members are sutured to the stent filaments along the intermediate portion.

Alternatively or additionally to any of the embodiments above, wherein the medical device further includes a covering.

Alternatively or additionally to any of the embodiments above, wherein at least one of the plurality of tubular members is glued to the covering.

Another example medical device comprises:
a stent including a plurality of longitudinally extending filaments;
a plurality of tubular members extending along the stent, the plurality of tubular members each having a lumen extending therein;
one or more radioactive elements;
wherein each of the plurality of tubular members is coupled with one or more of the plurality of longitudinally extending filaments; and
wherein one or more radioactive elements is positioned inside the lumen of one or more of the plurality of tubular members.

Alternatively or additionally to any of the embodiments above, wherein the radioactive element is a radioactive seed, a radioactive strand or both.

Alternatively or additionally to any of the embodiments above, the radioactive element and a spacer is positioned inside one or more of the tubular members, and wherein the radioactive element is positioned adjacent the spacer.

Alternatively or additionally to any of the embodiments above, further comprising a plurality of radioactive elements and a plurality of spacers located inside one or more of the plurality of tubular members, wherein at least one of the plurality of spacers is positioned adjacent each of the plurality of radioactive elements.

Another example medical device comprises:
a stent having one or more longitudinally extending filaments braided together;
a plurality of tubular members interwoven with the braided filaments, wherein each of the tubular members has a lumen extending therein; and
a plurality of radioactive strands positionable inside the lumens of the plurality of tubular members, wherein each radioactive strand includes radioactive seeds, and a spacer interposed between adjacent radioactive seeds.

The above summary of some embodiments is not intended to describe each disclosed embodiment or every implementation of the present disclosure. The Figures, and Detailed Description, which follow, more particularly exemplify these embodiments.

### BRIEF DESCRIPTION OF DRAWINGS

The disclosure may be more completely understood in consideration of the following detailed description in connection with the accompanying drawings, in which:

FIG. 1 is an example stent including tubular members, radioactive elements and spacers.

FIG. 2 is an example radioactive element.

FIG. 3 is an example radioactive strand having radioactive seeds and spacers.

FIG. 4 is an example tubular member including radioactive elements and spacers.

FIG. 5 is an example stent including tubular members, radioactive elements and spacers.

FIG. 6 is a cross section of an example radioactive stent and tubular member.

FIG. 7 is an example stent including tubular members, radioactive elements and spacers.

FIG. 8 is an example stent including tubular members, radioactive elements and spacers.

FIG. 9 is an example stent including an example shield positioned on the outside of the stent.

FIG. 10 is an example stent including an example shield positioned within a strut of the stent.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Treatment of abnormal tissue growth (e.g. cancer) may be accomplished through a variety of methodologies. For example, treatment of cancer may include the placement and deployment of a stent across the diseased tissue. However, in some instances stenting outcomes may be improved by combining one or more conventional therapies. For example, combining stent placement with radiation therapy may improve cancer treatment outcomes as compared to either stent or radiation therapy alone. Therefore, it may be desirable to utilize materials and/or design a stent that combines traditional stenting with radiation therapy. Some of the examples and methods disclosed herein may include a stent that can delivery radiation therapy.

Stents disclosed herein may treat esophageal cancers. Additionally, the stent may treat other forms of disease, including gastrointestinal, airway, urethra, ureter, cardiac, brain, breast, bladder, kyphoplasty and peripheral vascular disease, for example. Further, the stents disclosed herein may also be used in excisional cavities in solid and/or hollow organs.

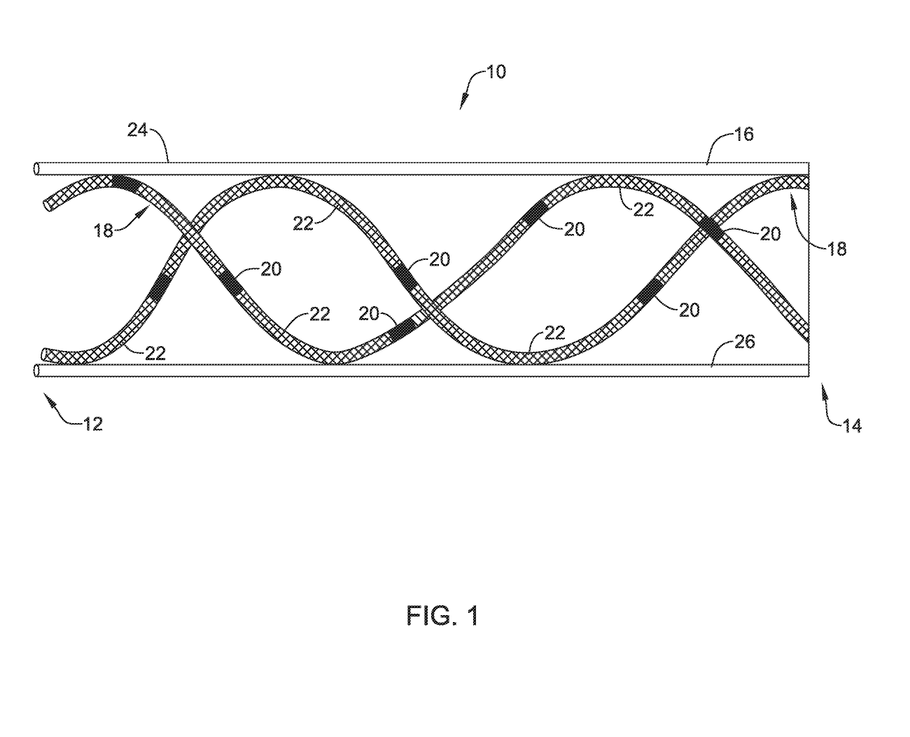

FIG. 1 shows an example radioactive stent system 10. Stent system 10 may include a stent 16 and one or more tubular members 18. Tubular members 18 may include one or more of a variety of radioactive elements 20. The radioactive elements 20 may be separated from each other by one or more spacers 22. As will be discussed in greater detail below, tubular members 18 may extend longitudinally along stent 16. While FIG. 1 shows tubular members 18 extending along the entire length of stent 16, in other examples, the tubular members 18 may extend only along a part of stent 16.

In some instances, stent 16 may be a self-expanding stent. Self-expanding stent examples may include stents having one or more filaments combined to form a rigid and/or semi-rigid stent structure. For example, stent filaments may be braided, intertwined, interwoven, weaved, knitted or the like to form the stent structure. Self-expanding stents may be manufactured from a single, cylindrical tubular laser-cut Nitinol members.

In other instances stent 16 may be a balloon expandable stent. Balloon expandable stents may be manufactured from a single, cylindrical tubular member. For example, in some instances, a cylindrical tubular member may be laser cut to form a balloon expandable stent.

Stent 16 in examples disclosed herein may be constructed from a variety of materials. For example, stent 16 (e.g. self-expanding or balloon expandable) may be constructed from a metal (e.g., Nitinol). In other instances, stent 16 may be constructed from a polymeric material (e.g., PET). In yet other instances, stent 16 may be constructed from a combination of metallic and polymeric materials. Additionally, stent 16 may include a bioabsorbable and/or biodegradable material.

Stent 16 may include a covering. For example, stent 16 may be partially or fully covered by an elastomeric or non-elastomeric material. Additionally, stent 16 may be partially or fully covered by a polymeric material such as silicone or ePTFE. Further, the covering (e.g., polymer) may span the spaces (e.g., openings, cells) in the wall of stent 16. In some examples, the covering may be applied by spraying, dipping, spinning or attaching a polymer sheet or tube the inner and/or outer surface of stent 16. In some examples, the covering may cover the stent filaments, tubular members 18 or both the stent filaments and the tubular members 18. Further, in some examples, the covering may cover a combination of one or more of the stent filaments and one or more of the tubular members 18. Additionally, in other examples the stent filaments and/or the tubular members 18 may extend partially or all the way through the covering.

In some examples, stent 16 may include anti-migration elements. Anti-migration elements may include flares, fins, micro-patterns, controlled ingrowth features, quills, or the like. Anti-migration features may be beneficial in controlling the amount stent 16 moves during and/or after deployment in the lumen. In some instances, the stent filaments and/or tubular members may include quills to prevent stent migration as described in U.S. Pat. No. 8,715,334, the entirety of which is fully incorporated herein.

In some instances, it may be favorable to ensure that the radioactive seeds are positioned inside the stent in order to minimize the occurrence of “hot spots” at the tissues contacting the stent near the seeds. This can be accomplished by positioning the tubular members inside the stent or by ensuring that the seeds are positioned inside the stent when the tubular members are positioned over and under the stent filaments.

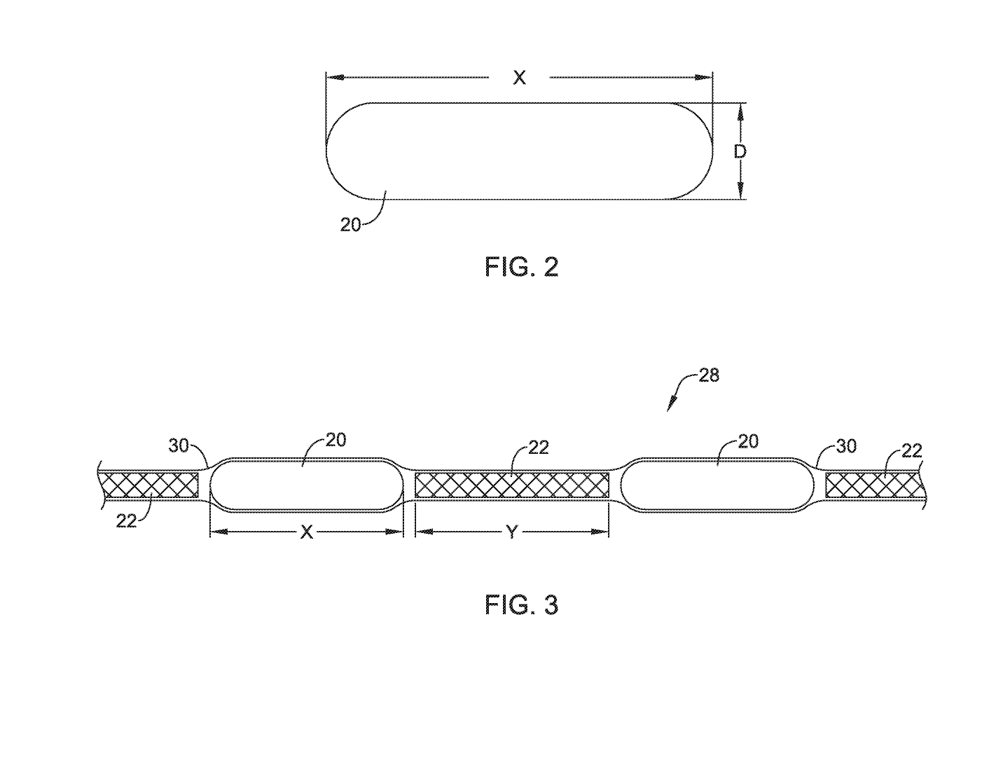

FIG. 2 shows an example radioactive element 20. In some instances, radioactive element 20 may be referred to as a “seed.” The terms “radioactive element” and “seed” may be used interchangeably throughout the remainder of this discussion. In general, seed 20 may be positioned adjacent a target site, whereby seed 20 may release radioactive energy and/or material, thereby radioactively treating the target location.

Seed 20 may be generally shaped as shown in FIG. 2. In other words, seed 20 may be an elongated cylinder having rounded ends. However, other shapes are contemplated. For example, seed 20 may be rounded, ovular, rectangular, triangular, or the like.

FIG. 2 shows the length of seed 20 depicted as dimension “X” and the diameter of seed 20 as dimension “D.” Depending on the particular therapeutic application, different types of seeds may have different dimensions. For example, in some instances, seed 20 may have a length “X” of between 1 and 20 mm. In other examples, seed 20 may have a length “X” between 2 and 10 mm, or between 3 and 8 mm. In some examples, seed 20 may have a length of about 5 mm.

Additionally, in some instances, seed 20 may have a diameter “D” of between 0.1 and 1.5 mm. In other examples, seed 20 may have a diameter “D” between 0.2 and 1 mm, or between 0.3 and 0.8 mm. In some examples, seed 20 may have a diameter of about 0.5 mm.

Seed 20 may include a variety of radioactive materials and or combinations of various materials. For example, seed 20 may include Iodine-125 (e.g. GE Oncura THINSeed™, IsoAid Advantage™ by IsoAid, Best™ Iodine-125), Palladium-103 (e.g. CivaString™ by CivaTech Technology, Theraseed™ by Theragenics, Best™ Palladium-103), Cesium-131, Gold-198, Iridium-192 and/or Ytterbium-169 or any other variations and/or derivatives thereof. Further, seed 20 may include other types of radioactive material. Additionally, seed 20 may include beta-emitting radionuclides.

For at least some examples disclosed herein, it is contemplated that one or more different radioactive elements 20 may be combined with one another to target a desired therapeutic outcome. For example, one or more of the radioactive materials disclosed above may be combined with one another to target a desired therapeutic outcome. Additionally, it is contemplated that different radioactive elements 20 having different radioactivity properties may be combined.

In some instances, one or more seeds 20 may combined with one or more additional seeds 20 and/or one or more spacing elements to form an elongated treatment member. For example, FIG. 3 shows elongated treatment member 28 including seeds 20 and spacing elements 22. In some instances (including the following discussion herein), treatment member 28 may be referred to as a “strand.”

The example shown in FIG. 3 depicts a covering 30 surrounding the seeds 20 and spacers 22. In some instances, covering 30 may include a material capable of being placed over the combination of seeds 20 and/or spacers 22 to form a continuous strand 28. In some examples, covering 30 may include one or more of a variety of shrink tubing (e.g. a polymeric tubing capable of reducing in size upon the application or heat, for example). In other examples, the covering may include a bioabsorbable and/or biodegradable material. Additionally, in some instances seeds 20 and/or spacers 22 may be connected to one another via a bioabsorbable connector. In other words, a combination of seeds 20 and/or spacers 22 may be “linked” to one another by a bioabsorbable and/or biodegradable material. In some instances, the radioactive strand may include a radioactive wire.

Seeds 20 and spacers 22 may be spaced and/or distributed in various patterns and/or distributions along strand 28. The length of the spacers 22 (which may correspond to the space between any two seeds 20) may vary depending on the particular strand 28 configuration. Similarly, the length of a given seed 20 in combination with a variety of lengths of given spacers 22 may vary depending on a particular strand 28 configuration. For example, FIG. 3 depicts the length of an example seed 20 as “X” and the spacing distance between seeds as “Y.” In some example strands 28, the length “X” of the seed 20 may be between 2-8 mm, while the length “Y” of spacer 22 may be between 12-18 mm.

However, different lengths of the both seeds 20 and spacers 22 are contemplated. Further, it can be appreciated that while some examples depicted in the figures disclosed herein show each seed 20 separated by a spacer 22, in some stances one or more seeds 20 may be placed directly adjacent one another. For example, 2, 3, 4, 5, 6, 7, 8, 9, 10, 15, 20 or more seeds 20 may be placed adjacent one another in a given strand 28. Further, adjacently placed seeds 20 may be separated from other adjacently placed seeds 20 by any length spacer 22.

Additionally, a given seed 20 and a given spacer 22 may have different dimensions despite being positioned adjacent one another in a given strand 28. For example, a given strand 28 may have a variety of seeds 20 having a variety of different lengths, diameters and materials. Similarly, a given strand 28 may have a variety of spacers 22 having a variety of different lengths, diameters and materials. Further, it is contemplated that a given strand may combine seeds 20 and spacers 22 in a variety of different combinations, patterns, distributions, separations, arrangements, or the like depending on the particular strand design required for a particular therapeutic application or user preference, for example.

As discussed above with respect to FIG. 1, in some instances it may be desirable to combine seeds 20 and/or spacers 22 with stent 16 to form a stent system 10 having the structural elements of stent 16 combined with the therapeutic properties of a radioactive material (e.g. seeds 20). Further, in some instances it may be desirable to utilize a structural element that can both engage with the stent structure while also being capable of accepting (e.g. holding) the seeds 20.

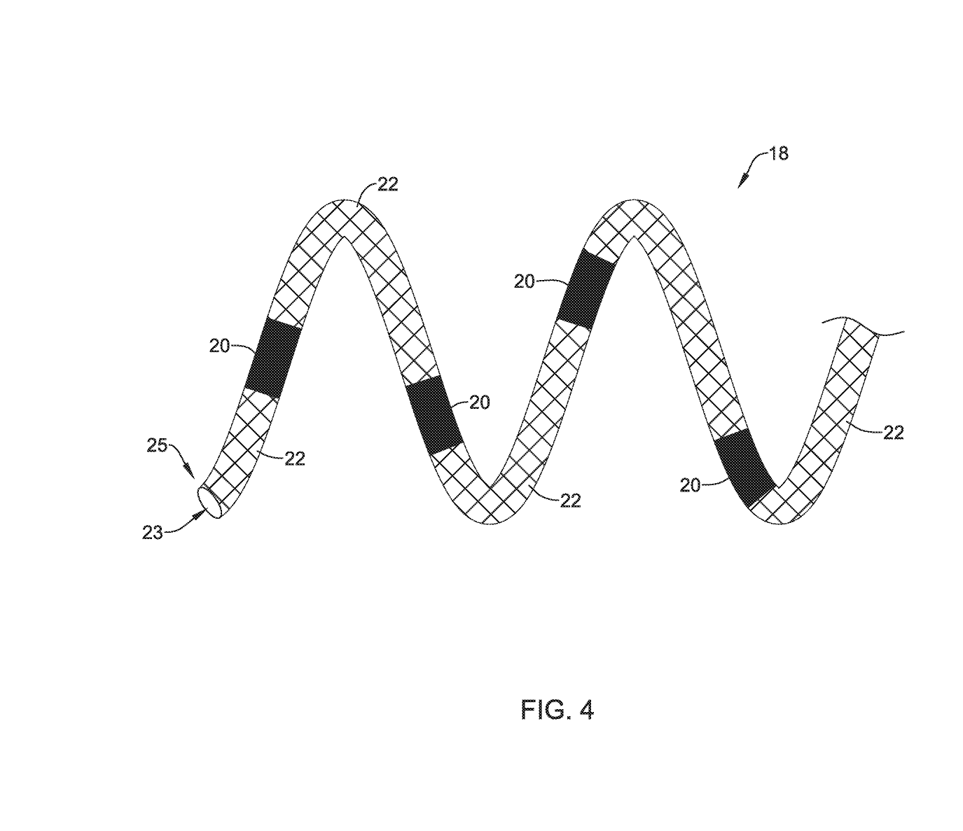

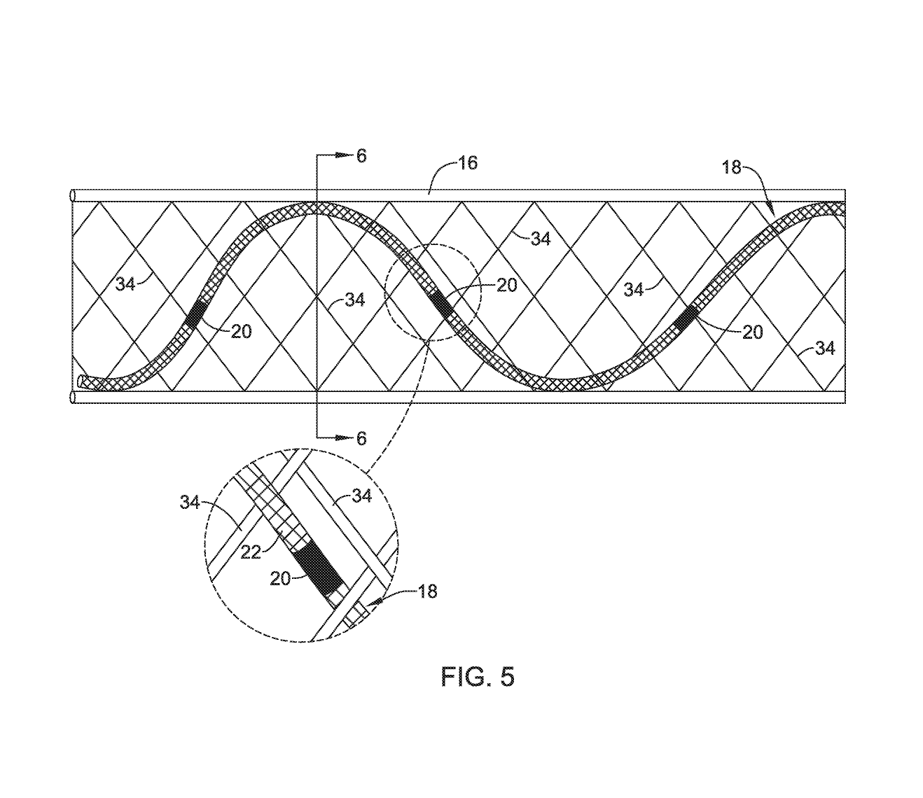

FIG. 4 shows an example tubular member 18 configured to accept, receive, hold and/or contain radioactive material (e.g. seeds 20) and/or spacers 22. While tubular member 18 is shown as generally helical in shape in one embodiment depicted in FIG. 4, this is not intended to be limited to a helical shape in other instances. For example, tubular member 18 may include a variety of shapes and/or configurations designed to engage and/or extend along stent 16.

As shown in FIG. 4, tubular member 18 may include lumen 23 designed to accommodate the placement of seeds 20, spacers 22 and/or a strand 28 within lumen 23 of tubular member 18. The process of placing seeds 20, spacers 22 and/or strands 28 inside tubular member 18 may be referred to as “loading” tubular member 18. Lumen 23 may extend along the entire length of the tubular member 18 (e.g. from a proximal portion to a distal portion).

In some instances, loading the seeds 20, spacers 22 and/or strands 28 into lumen 23 may be accomplished by pushing the seeds 20, spacers 22 and/or strands 28 directly into lumen 23. In other instances, loading the seeds 20, spacers 22 and/or strands 28 into lumen 23 may be accomplished by pulling the seeds 20, spacers 22 and/or strands 28 into lumen 23. For example, in some instances a strand 28 may include a pull wire designed to be inserted into one end of a tubular member 18 (e.g. through lumen 23) such that it can be seized at the opposite end of the tubular member 18. The seeds 20, spacers 22 and/or strands 28 may then be pulled (e.g. loaded) into lumen 23 via the pull wire. In some instances, the pull wire may be rounded and/or coated with a friction-reducing coating to ease its movement through lumen 23. Additionally, the pull wire may be constructed from a variety of materials. For example, the pull wire may be metallic or polymeric.

In some instances, it may be desirable to integrate tubular member 18 with stent 16 prior to the loading of the radioactive material (e.g. seeds) into lumen 23 of tubular member 18. For example, in some examples, one or more tubular members 18 may be combined and/or engaged with stent 16 through a distinct manufacturing process during which radioactive material is not integrated with the stent system (e.g. loaded into lumen 23 of tubular members 18) until immediately before insertion into the vasculature.

FIG. 5 shows example stent 16 engaged with one example tubular member 18. While FIG. 5 shows one tubular member 18, it is contemplated more than one tubular member 18 may be engaged with stent 16. For example, 2, 3, 4, 5, 6, 7, 8, 9, 10, 15, 20 or 50 tubular members may be coupled with stent 16. Further, as discussed above, FIG. 5 shows seeds 20, spacers 22 and/or strand 28 loaded into the tubular members 18. As shown, spacers 22 may be a variety of lengths, thereby creating a variety of patterns, arrangements and/or distributions of seeds 20.

In addition, FIG. 5 shows stent 16 including one or more longitudinally extending filaments 34. As discussed above, longitudinally extending filaments 34 may combine to form a self-expanding stent. For example, longitudinally extending filaments 34 may be braided, intertwined, interwoven, weaved, knitted or the like to form a self-expanding stent. Further, FIG. 5 shows that tubular members 18 may be integrated (e.g. intertwined) with the braided/weaved/knitted filaments 34 of stent 16. In other words, tubular members 18 may be one element in the overlapping structure that defines a braided stent 16. The detailed view 5A shows tubular member 18 (including seed 20 and spacer 22) braided with filaments 34. In other words, the tubular members 18 may be interwoven with the filaments 34 such that at some cross-over points the tubular member 18 is located radially outward of the filament 34 which the tubular member 18 crosses over, and at other cross-over points the tubular member 18 is located radially inward of the filament 34 which the tubular member 18 crosses over. In some instances, such as those examples in which the stent includes a covering, the tubular members may extend through a portion or all the way through the covering.

While FIG. 5 shows one tubular member 18 braided with one or more stent filaments 34, it is contemplated that more than one tubular member 18 may be utilized to construct the braided structure. Further, in some instances tubular members 18 may be partially braided with one or more stent filaments 34.

In other examples, tubular members 18 may be intertwined, interwoven, weaved, etc. within the structure (e.g. braided filaments, covering) of stent 16 without being a component of the braided stent structure or the covering. For example, tubular members 18 may be wound helically (clockwise, counterclockwise, or both) along the inside, outside or both the inside and outside surfaces of stent 16. Tubular members 18 may follow (e.g. extend alongside) one more filaments and/or a covering of stent 16. In other examples, the tubular members 18 may extend generally straight (e.g. longitudinally) along the inside, outside or both the inside and outside surfaces of stent 16.

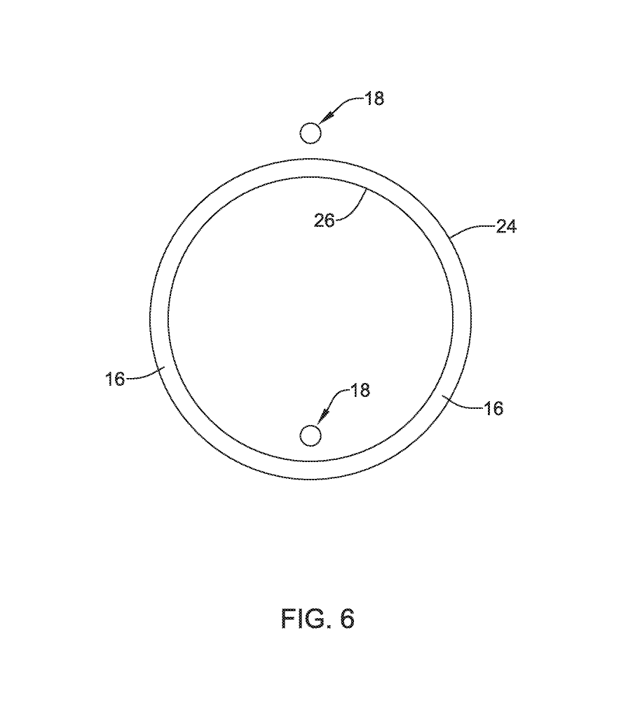

Further, in any configuration the tubular members 18 may weave from an inside surface of stent 16 to an outside surface of stent 16, then back to an inside surface of stent 16, and so on. In other words, tubular members 18 may extend from a position inside stent 16, through an opening in stent 16 to a position outside stent 16, back to a position inside stent 16 through another opening in stent 16, and so on. FIG. 6 shows a cross sectional view along line 6-6 of FIG. 5. In FIG. 6, tubular member 18 may be positioned on the outer surface 24 of example stent 16. Additionally, a portion of tubular member 18 may remain positioned “inside” example stent 16. For example, example tubular member 18 may be positioned on the inner surface 26 of stent 16. The particular descriptions of the patterns for which tubular members may extend along stent 16 are not intended to be limiting, rather, it is contemplated that a variety and/or combinations of patterns may be utilized that couple stent 16 and tubular members 18. Additionally, as stated above tubular members 18 may extend through stent openings as described above, while additionally extending through a covering coupled to the stent 16.

In some instances, tubular members 18 may be coupled to stent 16 using alternative and/or additional methods as those already described herein. For example, tubular members 18 may be sutured to individual stent filaments 34. The sutures may include longitudinal members that wrap around both a tubular member 18 and one or more stent filaments 34. The location of the sutures may be at a “cross-over” point of one or more filaments 34 and/or tubular members 18. In other words, a suture may extend around one or more filaments 34 and tubular members 18 in any combination. Further, the sutures may be positioned along the inner surface, the outer surface or both the inner and outer surfaces of stent 16. Additionally, the sutures may be constructed of a bioabsorbable and/or biodegradable material.

In other instances, tubular members 18 may be glued to individual stent filaments 34 or to the covering of the stent. The glue may include a polymer (e.g., silicone) that couples both a tubular member 18 and one or more stent filaments 34 and/or the stent covering. The location of the glue points may occur at “cross-over” points of one or more filaments 34 and/or tubular members 18. In other words, a suture may extend around one or more filaments 34 and tubular members 18 in any combination.

For covered stents, the glue may extend along the entire length of the tubular members. However, in some examples attaching the tubular members to the stent may include utilizing a covering mandrel having helical grooves. The covering mandrel may be used to insert the tubular members in the helical grooves. The stent may then be placed over the covering mandrel and the tubular members. The stent and the tubular members may then be covered with a polymer (e.g., silicone) by a dipping, spraying or other similar process.

In some instances, it may be desirable to load the seeds 20, spacers 22 and/or strands 28 into the tubular members 18 after the tubular members 18 have been integrated with stent 16 (e.g. via braiding, weaving, suturing, gluing, etc. as described above). In other instances, the seeds 20, spacers 22 and/or strands 28 may be loaded into the tubular members 18 after the stent has been implanted in the lumen. This may be accomplished through the use of an endoscope, for example.

Additionally, in some examples seeds 20, spacers and/or strands 28 may be “replaced” within tubular members 18. In other words, it is contemplated that a seed 20, spacer 22 and/or strand 28 may be individually removed and replaced by another seed 20, spacer 22 and/or strand 28. The replacement seed 20, spacer 22 and/or strand 28 may be the same or a different material (e.g., radioactive material). In some instances, replacing the radioactive material may alter and/or change the isotopes. Replacing the radioactive source may be accomplished before or after the medical device (e.g. stent system 10) has been deployed at a target location. Examples of replacement of radioactive elements may include those discussed in U.S. Patent Publication No. 20150190654, the entirety of which is incorporated herein.

As discussed above, the arrangement, pattern and/or distribution of seeds 20 may be varied along the length of stent 16. For example, by varying the distances between the seeds 20 (e.g. by varying the length of the spacers 22), the overall distribution of seeds 20 along both a circumferential and a longitudinal direction can be varied. The distribution of the tubular members 18 and, therefore, seeds 20, may be symmetrical or asymmetrical along any direction of stent 16.

Creating variations in the pattern of seeds 20 may be accomplished by changing both structural elements of the stent system and/or the spacing between the structural elements. For example, increasing the number of tubular members 18 engaged to a given stent 16 may result a more dense number of radioactive seeds 20 for a given circumferential surface of stent 16. Furthermore, it can be appreciated that an increased density may result from increasing the total number of radioactive seeds in a given tubular member (e.g. via reducing the length of spacers 22, thereby allowing the greater number of seeds loaded within a given tubular member 18). In some instances, the distribution of seeds along stent 16 may be such that the tissue surrounding stent 16 may receive a substantially uniform amount of radioactive energy. In other instances, tubular members 18 may be asymmetrically arranged about stent 16 such that a concentrated amount of radiation is delivered to a specific target tissue location. For example, an asymmetrically shaped tumor may require an asymmetrical distribution of tubular members 18 (and therefore, a non-uniform distribution of radioactive seeds 20) configured to deliver a customized dose of radiation to the tissue of the asymmetrical tumor.

Further, it is contemplated that radioactive seeds 20 having different radioactivity may be positioned along specific portions of stent 16. For example, seeds 20 having higher radioactivity may be positioned adjacent to the ends of a stent 16 while seeds 20 having relatively lower radioactivity may be positioned away from the ends of stent 16 (e.g., along a central portion of stent 16). In other examples, seeds 20 having lower radioactivity may be positioned adjacent to the ends of a stent 16 while seeds 20 having relatively higher radioactivity may be positioned away from the ends of stent 16 (e.g., along a central portion of stent 16). Thus, in some instances one or more seeds 20 having a first radioactivity and/or half-life may be placed in a tubular member 18 at a first end region of the tubular member 18, followed by one or more seeds 20 having a second radioactivity and/or half-life at a central region of the tubular member 18, followed by one or more seeds 20 having the first radioactivity and/or half-life (or a third radioactivity and/or half-life) at a second end region of the tubular member 18. The first radioactivity and/or half-life may be different from the second radioactivity and/or half-life and/or the third radioactivity and/or half-life, such as greater than or less than the second radioactivity and/or half-life and/or the third radioactivity and/or half-life. This arrangement may be repeated for each tubular member 18 arranged about stent 16, if desired. Specific (e.g., custom) arrangement of seeds 20 along stent 16 may improve dose distribution.

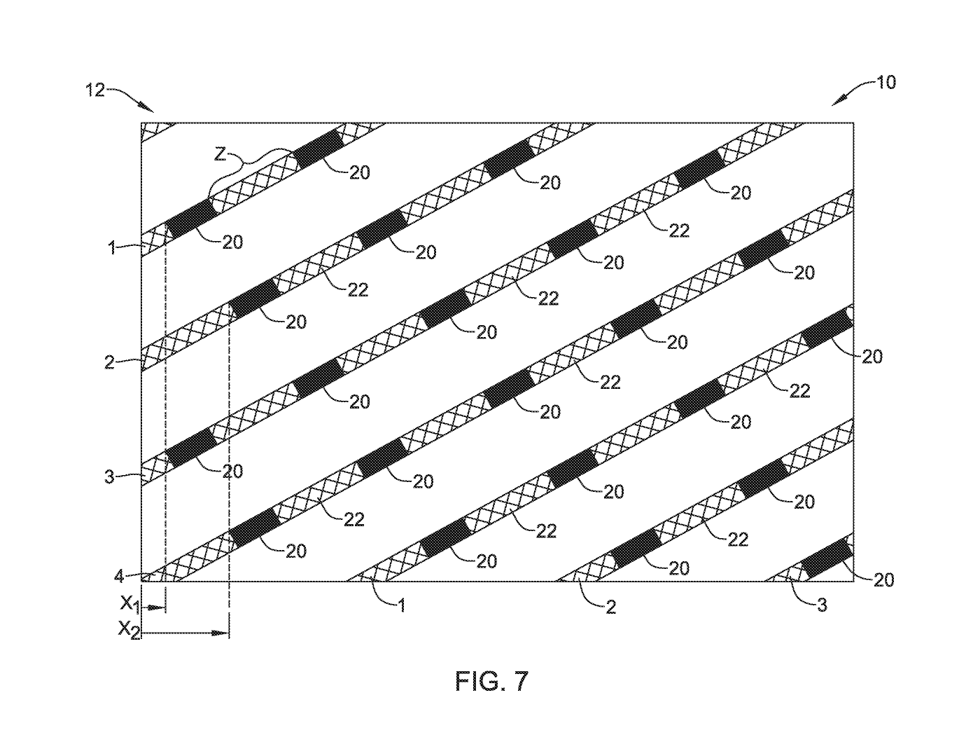

FIG. 7 shows an example stent system 10 similar to examples described above (e.g. a stent including one or more tubular members, radioactive elements and/or spacers) viewed as a flat pattern (e.g. a stent system as described herein cut along its longitudinal axis and laid flat). As can be seen in FIG. 7, four tubular members (labeled 1-4 in FIG. 7) are engaged longitudinally along stent 16 in a helical arrangement. In some examples, the number of tubular members 18 in stent system may include more or less than four members 18. For example, in some instances stent system 10 may include 1, 2, 3, 4, 5, 6, 7, 8, 9, 10, 15, 20 or more tubular members 18. In one example stent system 10, the number of tubular members may include six tubular members 18.

When viewed as a flat pattern, the four tubular members 18 are substantially parallel and spaced approximately equidistant from one another. Further, if the stent shown in FIG. 7 were viewed as a cylinder (e.g. as it would be when delivered to a target site in the lumen), tubular members 1-4 would wrap around stent 16 as parallel helices. It is understood that example stent system 10 may include more or less than four tubular members 18.

FIG. 7 shows stent 16 having a proximal end 12. Further, each tubular member 1-4 includes a seed 20 that is closer to proximal end 12 than any of the other seeds 20 in the respective tubular member 18. Moreover, FIG. 7 shows that for each tubular member 1-4, the “most proximal” seed 20 may be “offset” from the proximal end 12 of the stent 15 by a given distance. For example, the most proximal seed 20 of first tubular member 1 has a proximal offset defined as X1. Similarly, the most proximal seed 20 of the second tubular member 2 has a proximal offset X2, which is different from proximal offset X1 of most proximal seed 20 of first tubular member 1. As shown in FIG. 7, the proximal offsets of each of third and fourth tubular members 3 and 4 are substantially equivalent to the proximal offsets of first and second tubular members 1 and 2, respectively. In some example, proximal offset X1 may 1 mm to 10 mm, or about 3 mm to 7 mm. In other examples, proximal offset X1 may be about 5 mm. In some examples, proximal offset X2 may be about 10 mm to 30 mm, or about 15 mm to 25 mm, or about 18 to 22 mm. In other examples, proximal offset X2 may be about 20 mm.

Further, the spacing between seeds 20 may be adjusted to vary the overall pattern, distribution and/or density of the radioactive elements along stent 16. As shown in FIG. 7, the space between the first two seeds corresponding to first tubular member 1 (e.g. the length of an example spacer) is labeled “Z.” In some example, distance “Z” may be about 5 mm to 40 mm, or about 10 mm to 30 mm, or about 15 mm to 25 mm, or about 18 mm to 22 mm. It can be appreciated that the lengths of the spacers and proximal offsets can be varied to achieve many different variations in the overall distribution of radioactive material along stent 16.

In some examples (such as the example described with respect to FIG. 7), one or more seeds 20 may overlap when viewed along the longitudinal axis. In other words, in some instances the distal (or proximal) end of a given seed 20 may overlap (longitudinally) with the proximal (or distal) end, respectively, of a different seed 20. As can be appreciated, longitudinal overlapping seeds 20 may occur in stent designs having a greater density, and hence, closer spaced seeds 20. In other examples, the distal/proximal end of a given seed 20 may not overlap (longitudinally) with the proximal/distal end, respectively, of any other seed 20.

In addition, tubular members 18 may be also be adjusted by varying the braid angle and/or the degree at which a given tubular member “starts” with respect to the proximal end 12 of the stent. For a braided stent, it may be desirable to have the tubular members 18 at the same angle as the stent filaments in order to allow for the stent to be compressed in the delivery device, since a mismatch of the braid angle may prevent compression of the stent.

Further, in some instances a strand 28 may be constructed of seeds 20 and spacers 22 alternating along the longitudinal axis of stent 16. In one example, seeds and spacers 20/22 may alternate every other along the length of stent 16 and may include seeds 20 from 2 to 8 mm in length and spacers from 12 to 18 mm in length. For example, one arrangement may have seeds 20 that are 5 mm in length alternating with spacers 22 that are 15 mm in length.

Further, in other examples, a plurality of tubular members 18 included in a given stent system may have one “grouping” of tubular members that have a proximal offset and stent/spacer 20/22 arrangements that are different from a second “grouping” of tubular members. For example, in some examples, a first grouping of tubular members 18 may include a proximal offset of approximately 2 to 7 mm (e.g. 5 mm), while the second grouping of tubular members 18 may include a proximal offset of approximately 17 to 23 mm (e.g. 20 mm).

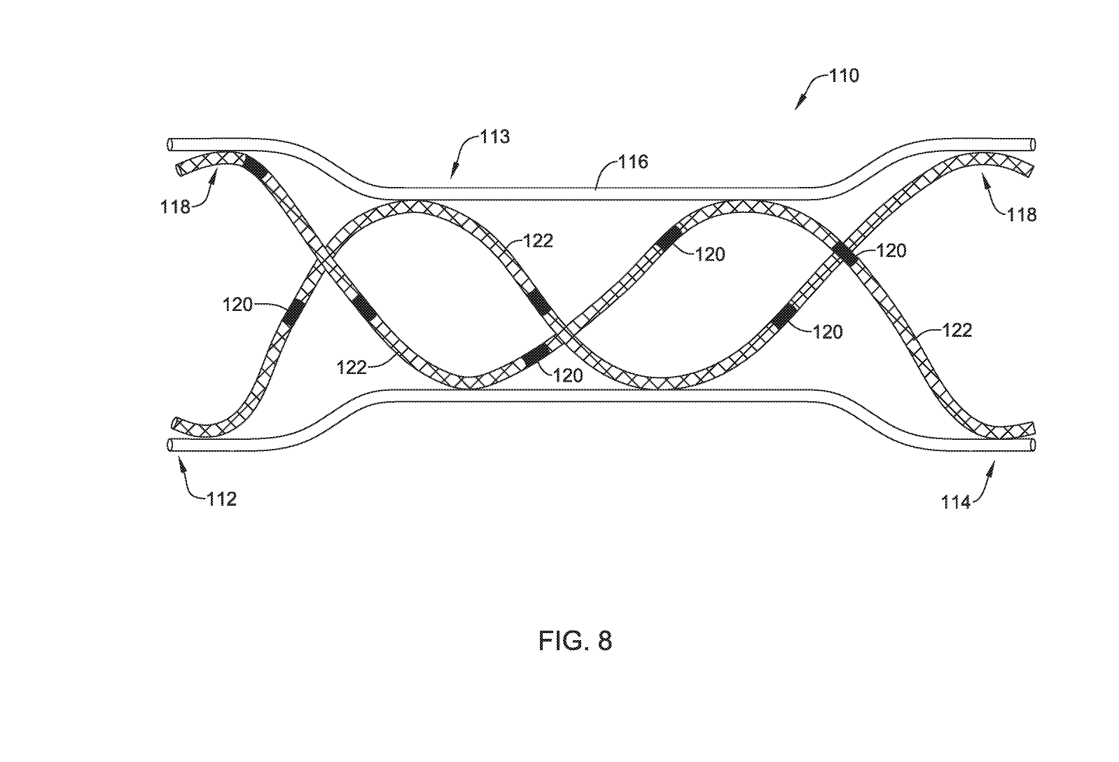

FIG. 8 shows an alternative stent system 110. Stent system 110 may be similar the stent system 10 discussed above with respect to FIG. 1. For example, stent system 110 may include stent 116 and one or more tubular members 118. Tubular members 118 may include one or more of a variety of radioactive seeds 120. The seeds 120 may be separated from each other by one or more spacers 122. As will be discussed in greater detail below, tubular members 118 may extend longitudinally along stent 116.

In some instances, stent 116 may be a self-expanding stent. Further, as shown in FIG. 8, stent 116 may have a proximal portion 112, a distal portion 114 and an intermediate portion 113. As shown, the proximal and distal portions 112/114 of stent 116 may be flared or enlarged relative to the intermediate portion 113, such that the proximal and distal portions 112/114 have a larger overall diameter than intermediate portion 113. In some instances, the shape of stent 116 may resemble that of a “dog bone,” for example. Further, tubular members 118 may be connected to the filaments (not shown) of stent 116 by sutures and/or glue along the proximal, distal and/or intermediate portions 112/114/113. Further, in other instances the tubular members may be connected to stent 116 along the intermediate portion 113, while not connected along either the proximal or distal portions 112/114.

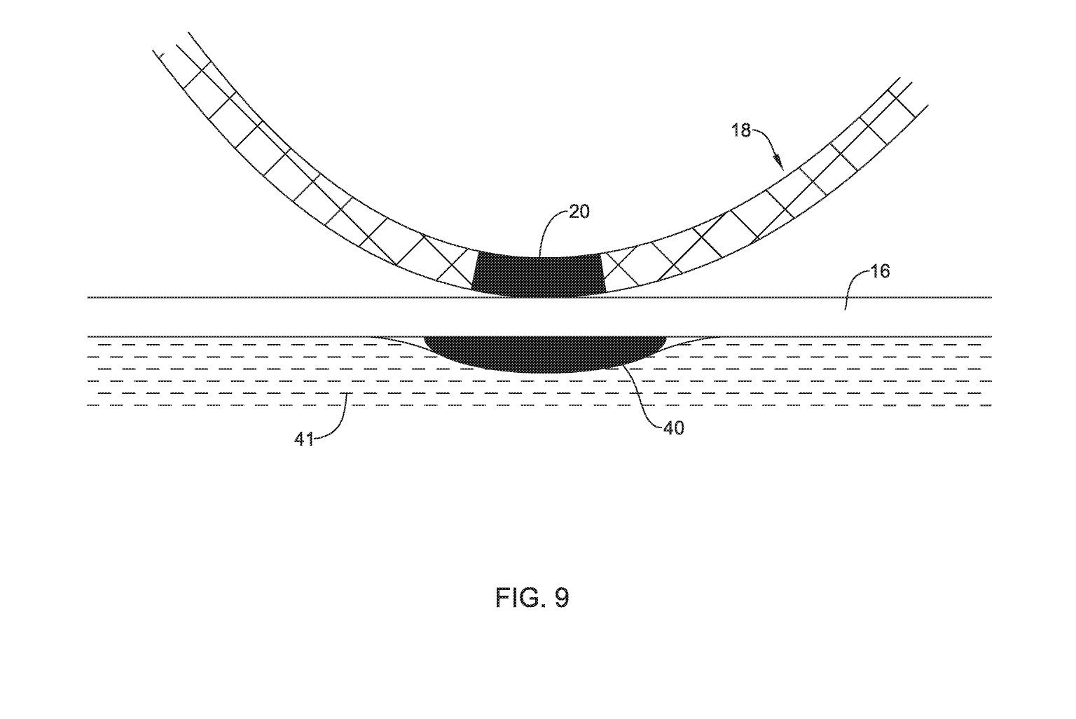

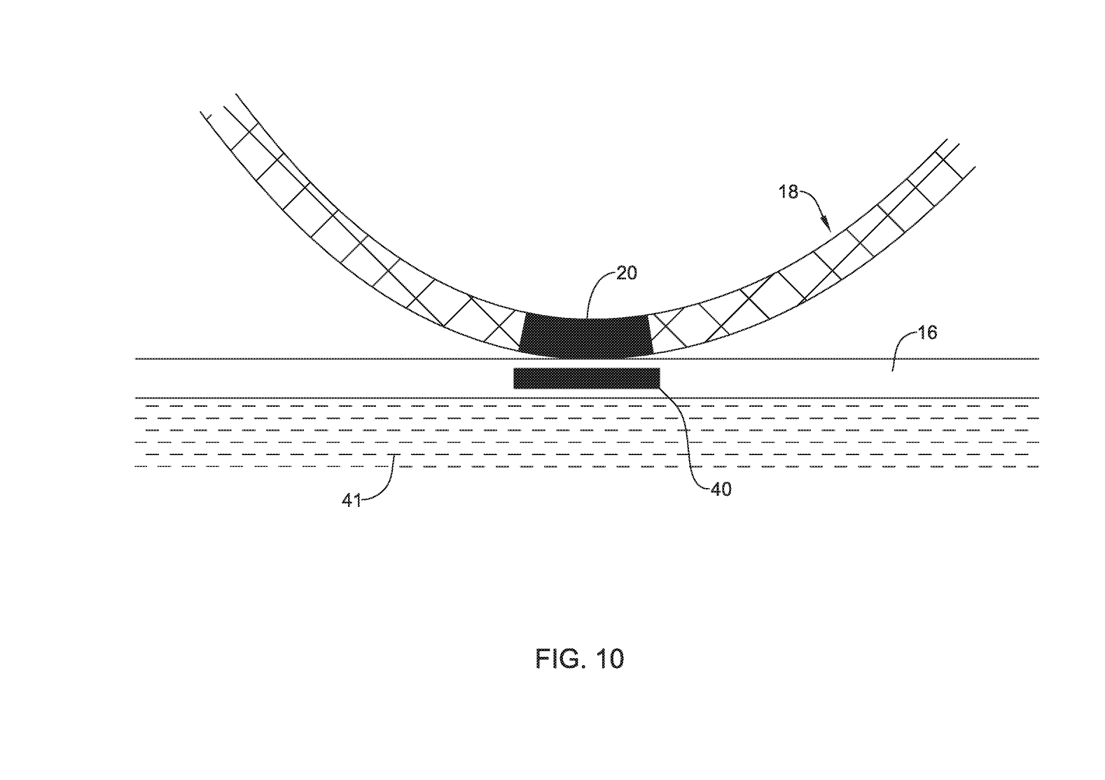

In some instances, the examples discussed herein may further include one or more “intensity modulation filters” (also referred to herein as “shields”) designed to reduce and/or modulate the amount of radiation delivered by a radioactive seed 20. For example, one or more shields may be placed between a radioactive seed 20 and the vessel wall (e.g. targeted tissue) in order to modulate the amount of radiation reaching the tissue. FIG. 9 shows shield 40 positioned between stent 16 and tissue 41. As shown in FIG. 9, in some instances one or more shields 40 may be placed on the outer surface of stent 16, thereby modulating the radiation delivered by seeds 20 positioned on an inner surface of stent 16.

In other instances, one or more shields 40 may be positioned within at least a portion of the wall of a strut of stent 16 and/or in the wall of a catheter and/or tubular member 18 holding radioactive seed 20. For example, FIG. 10 shows an example shield 40 positioned within at least a portion of the wall of a strut of stent 16. It can be appreciated that shield 40 may be completely embedded within the example stent strut of stent 16. However, it is further contemplated that a portion of shield 40 may extend beyond an inner surface and/or outer surface of the example stent strut of stent 16 and/or tubular member 18. In some instances, tubular members 18 may include one or more shielded regions, including one or more shields 40 along the length of tubular member 18. Shields 40 may be embedded in the wall of tubular member 18, inserted into lumen of tubular member 18, and/or positioned on an outer peripheral surface of tubular member 18, as desired.

Shields 40 may be constructed out of a variety of materials including metal, metallic powder, polymer, etc. and in some instances may be placed inside a polymer. For example, the shields may include tungsten powder inside silicone. Further, in some instances, shield 40 may be of varying thickness. In some examples the thickest portion of shield 40 may include that portion of the shield 40 that is closest to the seed. Further, the thickness may taper (and become thinner) at the shield extremities. Additionally, in some instances shields 40 may include one or more openings or holes (not shown in FIG. 9) extending fully or partially through the shield wall.

In some instances, shield 40 may be coupled to stent 16 and/or tubular members 18 by a variety of attachment methods (e.g. gluing, etc.). For example, in some instances the shield 40 may include a metal plate coupled to stent 16 and/or tubular members 18. In other instances, a shield may be applied by spraying, painting or similar methods. In some instances, a shield coupled to a tubular member 18 may not cover the entire circumference and/or length of the tubular member.

Materials that may be used for the various components of stent system 10 and the various examples disclosed herein may include those commonly associated with medical devices. For simplicity purposes, the following discussion makes reference to stent system 10. However, this is not intended to limit the devices and methods described herein, as the discussion may be applied to other similar systems and/or components of stent systems or devices disclosed herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device, comprising:
a stent including a plurality of longitudinally extending filaments, the stent having an inner surface and an outer surface;
a plurality of tubular members extending along the stent;
wherein each of the plurality of tubular members is coupled with one or more of the plurality of longitudinally extending filaments; and
wherein each of the plurality of tubular members is configured to accept a radioactive element, a spacer or both.

2. The medical device of claim 1, wherein one or more of the plurality of tubular members is interwoven with one or more of the plurality of longitudinally extending filaments.

3. The medical device of claim 1, wherein the plurality of longitudinally extending filaments are braided together, and wherein at least one of the tubular members is interwoven with the braided filaments.

4. The medical device of claim 1, wherein one or more of the longitudinally extending filaments and one or more of the plurality of tubular members are braided together.

5. The medical device of claim 1, wherein the longitudinally extending filaments are braided, and wherein one or more of the plurality of the tubular members extends helically in a clockwise, counter-clockwise or both a clockwise and counter-clockwise direction along the stent.

6. The medical device claim 1, wherein the plurality of tubular members includes a first group of tubular members having a first distribution of seeds positioned therein, and wherein the plurality of tubular members includes a second group of tubular members having a second distribution of seeds positioned therein, and where the first and second distributions of seeds are different.

7. The medical device of claim 6, wherein the first distribution of seeds includes a first seed, and wherein the second distribution of seeds includes a second seed, wherein the first seed is closer to a proximal end of the stent than the second seed.

8. The medical device of claim 7, wherein the first seed is approximately 5 mm away from the proximal end of the stent and wherein the second seed is approximately 5 mm from the proximal end of the stent.

9. The medical device of claim 1, wherein at least a portion of the plurality of tubular members extends along the inner surface of the stent.

10. The medical device of claim 1, wherein at least a portion of the plurality of tubular members extends along the outer surface of the stent.

11. The medical device of claim 1, wherein at least a portion of the plurality of tubular members extends from the inner stent surface to the outer stent surface through an opening in the stent.

12. The medical device of claim 1, wherein one or more of the tubular members are sutured, glued or both sutured and glued to one or more of the longitudinally extending stent filaments.

13. The medical device of claim 1, wherein the stent has a distal portion having an outer diameter, a proximal portion having an outer diameter substantially equal to the distal portion outer diameter, and an intermediate portion located between the distal and proximal portions, wherein the intermediate portion has an outer diameter less than the outer diameter of the proximal and distal portions, and wherein the tubular members are sutured to the stent filaments along the intermediate portion.

14. The medical device of claim 1, wherein the medical device further includes a covering.

15. The medical device of claim 15, wherein at least one of the plurality of tubular members is glued to the covering.

16. A medical device, comprising:
a stent including a plurality of longitudinally extending filaments;
a plurality of tubular members extending along the stent, the plurality of tubular members each having a lumen extending therein;
one or more radioactive elements;
wherein each of the plurality of tubular members is coupled with one or more of the plurality of longitudinally extending filaments; and
wherein one or more radioactive elements is positioned inside the lumen of one or more of the plurality of tubular members.

17. The medical device of claim 16, wherein the medical device further includes a shield.

18. The medical device of claim 16, wherein the radioactive element and a spacer is positioned inside one or more of the tubular members, and wherein the radioactive element is positioned adjacent the spacer.

19. The medical device of claim 18, further comprising a plurality of radioactive elements and a plurality of spacers located inside one or more of the plurality of tubular members, wherein at least one of the plurality of spacers is positioned adjacent each of the plurality of radioactive elements.

20. A medical device, comprising:
a stent having one or more longitudinally extending filaments braided together;
a plurality of tubular members interwoven with the braided filaments, wherein each of the tubular members has a lumen extending therein; and
a plurality of radioactive strands positionable inside the lumens of the plurality of tubular members, wherein each radioactive strand includes radioactive seeds, and a spacer interposed between adjacent radioactive seeds.
